(19) United States
(12) Patent Application Publication
(10) Pub. No.: US 2011/0319636 A1
(43) Pub. Date: Dec. 29, 2011

(54) METHOD FOR PREPARATION OF AMIDES FROM ALCOHOLS AND AMINES BY EXTRUSION OF HYDROGEN

(75) Inventors: Lars Ulrik Nordstrom, Copenhagen (DK); Henning Vogt, Aachen (DE); Robert Madsen, Kgs. Lyngby (DK); Johan Hygum Dam, Odense (DK)

(73) Assignee: Technical University of Denmark

(21) Appl. No.: 13/130,693

(22) PCT Filed: Nov. 23, 2009
(86) PCT No.: PCT/EP2009/065641

§ 371 Date: Sep. 7, 2011

Related U.S. Application Data
(60) Provisional application No. 61/117,294, filed on Nov. 24, 2008.

(30) Foreign Application Priority Data
Nov. 24, 2008 (DK) PA200801645
Aug. 27, 2009 (DK) PA200900967

(51) Int. Cl. C07C 227/12; C07D 207/16

(52) U.S. Cl. 548/537; 564/182; 564/218; 564/166

(57) ABSTRACT

The present invention relates to a method for preparation of carboxamides using alcohols and amines as starting materials in a dehydrogenative coupling reaction catalyzed by a ruthenium N-heterocyciic carbene (NHC) complex, which may be prepared in situ.

### FIELD OF INVENTION

The present invention relates to a method for preparation of carboxamides providing a more direct, easy, economical and environmentally friendly synthesis of amides using alcohols and amines as starting materials in a dehydrogenative coupling reaction catalyzed by a ruthenium N-heterocyclic carbene (NHC) complex, which may be prepared in situ.

### BACKGROUND OF THE INVENTION

The amide bond is one of the most important linkages in organic chemistry and constitutes the key functional group in peptides, polymers and many natural products and pharmaceuticals [see eg. T. Cupido et al. Curr. Opin. Drug Discov. Dev. 2007, 10, 768-783; J. W. Bode, Curr. Opin. Drug Discov. Dev. 2006, 9, 765-775 and K. E. Gonsalves et al. Trends Polym. Sci. 1996, 4, 25-31]. Generally, amides are prepared from activated carboxylic acids and amines:

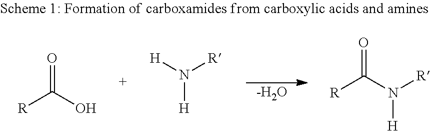

Amides are usually prepared by coupling of carboxylic acids and amines by the use of either a coupling reagent [S.-Y. Han et al. Tetrahedron 2004, 60, 2447-2467] or by prior conversion of the carboxylic acid into a derivative [C.A.G.N. Montalbetti et al., Tetrahedron 2005, 61, 10827-10852]. The activation of the carboxylic acid requires stoichiometric amounts of reagents, i.e. carboxylic acid and reagent in the ratio 1:1. The activating reagents can be expensive and will in all cases lead to the stoichiometric formation of by-products which must be separated from the product and disposed of as chemical waste.

Alternative procedures include the Staudinger ligation between esters and azides [M. Köhn et al. Angew. Chem. Int. Ed. 2004, 43, 3106-3116], aminocarbonylation of aryl halides [J. R. Martinelli et al. Angew. Chem. Int. Ed. 2007, 46, 8460-8463] and oxidative amidation of aldehydes [J. W. W. Chang et al. Angew. Chem. Int. Ed. 2008, 47, 1138-1140]. However, all these methods also require stoichiometric amounts of various reagents and lead to equimolar amounts of by-products and involve 2-3 chemical steps. In special cases, amides can be formed by catalytic procedures as demonstrated for the Schmidt reaction between ketones and azides [S. Lang et al. Chem. Soc. Rev. 2006, 35, 146-156], the Beckmann rearrangement of oximes [N. A. Owston et al. Org. Lett. 2007, 9, 3599-3601], and the amidation of thioacids with azides [R. V. Kolakowski et al. J. Am. Chem. Soc. 2006, 128, 5695-5702].

In the present invention a novel protocol for the synthesis of carboxamides is disclosed characterized by the direct amidation of both primary and secondary amines with alcohols in the presence of a homogeneous Ruthenium catalyst, which may be prepared in situ, resulting in liberation of hydrogen as the only by-product.

D. Milstein et al. disclosed a similar process wherein a Ruthenium pincer complex catalyzes the direct coupling of sterically unhindered primary amines and alcohols under homogeneous conditions [C. Gunanathan, Y. Ben-David, D. Milstein, Science 2007, 317, 790-792]. Milsteins protocol requires long reaction times (7-12 hours), however, and do not accept secondary amines. Furthermore, the catalyst employed by Milstein is highly sensitive, and must be prepared in advance using glove-box technology.

JP 56161359 discloses a method for preparation of amides by using alcohols and amines as reactants in the presence of a heterogeneous Ru-catalyst such as Ru metal supported on activated carbon, and in the presence of excess oxygen. Such conditions are known to promote the transformation of amines to their corresponding N-oxides, which themselves may lead to further by-products.

It was reported recently [Grützmacher et al., Angew. Chem. Int. Ed. (2009) 48 559] that the dehydrogenation can take place in the presence of a special rhodium catalyst. However, hydrogen is not liberated by the reaction and Grützmacher employs a stoichiometric amount of an alkene to remove the hydrogen. The alkene is apparently required in order for hydrogen to leave the rhodium catalyst, since the reaction does not proceed in the absence of the alkene as hydrogen acceptor.

A similar situation was also disclosed recently [Williams et al., Org. Lett., Vol. 11, No. 12, (2009) p. 2667]. Williams also prepares amides from alcohols and amines using a ruthenium catalyst, but like Grützmacher only in the presence of a stoichiometric hydrogen acceptor, in Williams' case a ketone, rather than an alkene. Williams employs only phosphine ligands, and not N-heterocyclic carbene ligands.

### SUMMARY OF THE INVENTION

A novel method for preparation of carboxamides from alcohols and amines is disclosed. The reaction is catalyzed by a ruthenium N-heterocyclic carbene (NHC) complex which may be prepared in situ from a suitable Ru(II) source, a phosphine and an azolium salt in a heated reaction mixture in the presence of a base, to which the alcohol and the amine substrates are subsequently added. After adding an optional solvent the reaction mixture is heated until deemed complete which usually requires up to a few hours.

The present invention thus in a first aspect relates to a method for the preparation of amides of formula (I) from alcohols of formula A and amines of formula B in the presence of a ruthenium complex of formula (IV):

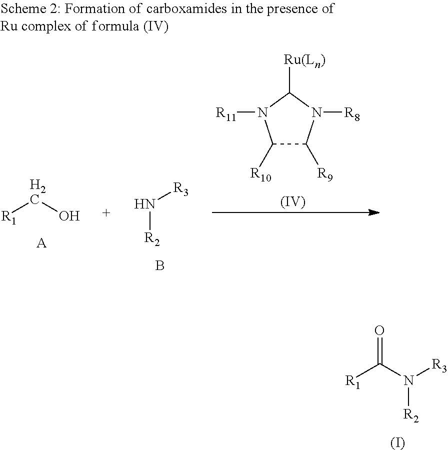

wherein
R₁ and R₂ are individually selected from the group consisting of hydrogen, aryl, heteroaryl, C₁-C₂₀ alkyl, alkenyl or aryl-C₁-C₄ alkyl, optionally substituted with one or more substituents selected from halogen, C₁-C₆ alkyl or C₁-C₆ alkoxy, and wherein R₁ and R₂ may be connected with a single bond,
R₃ is selected from hydrogen and C₁-C₄ alkyl,
R₈ and R₁₁ can be the same or different and are independently selected from the group consisting of C₁-C₆ linear or branched alkyl, C₃-C₆ cycloalkyl, aryl, aryl-C₁-C₄ alkyl, heteroaryl,
R₉ and R₁₀ are independently selected from the group consisting of hydrogen, C₁-C₆ linear or branched alkyl and aryl, and wherein R₉ and R₁₀ together with the heterocyclic ring they are attached to may form a 5-7 membered saturated or unsaturated ring optionally containing one or two heteroatoms selected from oxygen or nitrogen,
L is a ligand selected from phosphines, halides, C₁-C₆ alkoxides, arenes, alkylidine, vinylidine, indenylidine, alkenes, amines, pyridines, phosphine oxides and arsines,
n is 1 to 5
the dotted line denotes a single or double bond,
   which method comprises the steps of mixing the substrates of formula A and B in the presence of the catalyst of formula (IV), optionally adding a phosphine and/or a solvent, and heating the resulting reaction mixture until the reaction is deemed complete, followed by standard work-up and optional purification.

The present invention in another aspect relates to a method for preparation of carboxamides of the general formula (I):

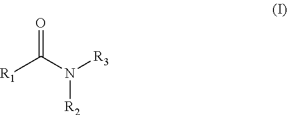

wherein R₁ and R₂ are individually selected from the group consisting of hydrogen, aryl, heteroaryl, C₁-C₂₀ alkyl or aryl-C₁-C₄ alkyl, optionally substituted with one or more substituents selected from halogen, C₁-C₆ alkyl or C₁-C₆ alkoxy, and wherein R₁ and R₂ may be connected with a single bond,
R₃ is selected from hydrogen and C₁-C₄ alkyl,
where said method comprises preparation of a catalyst, comprising heating a mixture of
a. a phosphine
b. an azolium salt
c. a base
d. a Ru (II)-source
e. optional solvent(s)
   for an appropriate time, followed by adding as substrates alcohols of formula A and amines of formula B as defined above, and continue heating until the reaction is deemed complete, followed by work-up and optional purification.

In one embodiment R₁ is phenyl, R₂ is C₁-C₂₀ alkyl and R₃ is hydrogen.

In another embodiment R₁ and R₂ are benzyl and R₃ is hydrogen.

In yet another embodiment R₁ is selected from 2-chlorophenyl, 3-chlorophenyl or 4-chlorophenyl; R₂ is benzyl and R₃ is hydrogen.

In yet another embodiment R₁ is phenyl, R₂ is benzyl and R₃ is hydrogen.

In one aspect of the invention, said method of preparation of the catalyst comprises the use of a phosphine with the general formula (II)

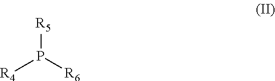

wherein R₄, R₅ and R₆ can be the same or different and are independently selected from the group consisting of C₁-C₆ linear or branched alkyl, C₃-C₆ cycloalkyl, aryl, aryl-C₁-C₄ alkyl, heteroaryl, biaryl wherein each aryl, biaryl and heteroaryl may optionally be substituted with aryl, C₁-C₄ alkyl, di(C₁-C₄ alkyl)amino and up to two halogen atoms selected from F, Cl and Br, and wherein two substituents selected from R₄, R₅ and R₆ may be fused and together with the phosphorous atom form a 5-7 membered ring.

In an embodiment R₄, R₅ and R₆ are all the same. In another embodiment two of R₄, R₅ and R₆ are the same. In a further embodiment R₄, R₅ and R₆ are all different.

In one preferred embodiment the phosphine is tricyclohexylphosphine, PCy₃. In another preferred embodiment the phosphine is tricyclopentylphosphine, PCyp₃. In a further preferred embodiment the phosphine is biphenyldicyclohexylphosphine (Cy-JohnPhos).

In a particular embodiment the phosphine is employed as its tetrafluoroborate (HBF₄) salt.

In another aspect of the invention said method of preparation of the catalyst comprises the use of an azolium salt with the general formula (III)

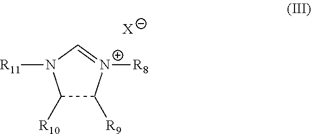

wherein X⁻ is Cl⁻, Br⁻; BF₄⁻, CF₃SO₃⁻ I⁻, C₁-C₆-alkyl-SO₃⁻, and (C₁-C₆ alkoxy)₂P(═O)O⁻ wherein R₈ and R₁₁ can be the same or different and are independently selected from the group consisting of C₁-C₆ linear or branched alkyl, C₃-C₆ cycloalkyl, aryl, aryl-C₁-C₄ alkyl, heteroaryl, and wherein R₉ and R₁₀ are independently selected from the group consisting of hydrogen, C₁-C₆ linear or branched alkyl and aryl,
and wherein R₉ and R₁₀ together with the heterocyclic ring they are attached to may form a 5-7 membered saturated or unsaturated ring optionally containing one or two heteroatoms selected from oxygen or nitrogen,
and wherein the dotted line denotes a single or double bond.

In another aspect of the invention the method of preparation of the catalyst comprises exchanging a ligand L in a ruthenium complex of formula (IV) with a phosphine of formula (II). In a specific embodiment of the invention the exchanged ligand L is cymene.

The method of preparation of the catalyst also comprises a base. In an embodiment of the invention the base is selected from the group consisting of alkali metal C₁-C₆ alkoxides, such as potassium tert-butoxide.

In another embodiment of the invention the base is selected from the group consisting of alkali metal carbonates, such as cesium carbonate.

In yet another embodiment the base is selected from alkali metal amides, such as potassium bis(trimethylsilyl)amide.

In a preferred embodiment the base is potassium tert-butoxide. In another preferred embodiment the base is potassium bis(trimethylsilyl)amide.

In a further embodiment between about 5 to about 30 mol % base is employed. In a specific embodiment the phosphine ligand is employed as a salt, and 20 mol % of base is employed. In another specific embodiment the phosphine ligand is employed as the free phosphine, and 15 mol % of base is employed.

In one embodiment the reaction is conducted in the absence of solvents. In another embodiment one or more solvents are added to the reaction mixture. Solvents which are suitable both for the preparation of the catalyst and for the dehydrogenative coupling of alcohols of formula A with amines of formula B comprise non-coordinating solvents selected from the group consisting of benzene, toluene, xylene, mesitylene, chlorobenzene, dichloromethane, carbon tetrachloride, dichloroethane, diethylether, dipropylether, di-butylether, methyl-tert-butylether (MTBE), tetrahydrofuran (TH F), methyltetrahydrofuran, 1,4-dioxane and 1,2-dimethoxyethane (DME) and mixtures thereof.

In a preferred embodiment the solvent is toluene.

In a further aspect of the invention, the method for preparation of the catalyst comprises the use of a ruthenium source. In an embodiment of the invention the ruthenium source is selected from the group consisting of Ru(PPh₃)₃Cl₂, Ru(alkene)Cl₂ wherein the alkene ligand is selected from norbornene, ethene, cyclooctene (COE) and cyclooctadiene (COD), and Ru(arene)Cl₂ wherein the arene ligand is selected from benzene, toluene, mesitylene, p-cymene and naphthalene.

In one preferred embodiment the ruthenium source is Ru(COD)Cl₂. In another preferred embodiment the ruthenium source is [Ru(p-Cymene)Cl₂]₂

In a further aspect of the present invention, the phosphine, the azolium salt and the ruthenium source react in the presence of the base to afford a ruthenium carbene with the formula (IV):

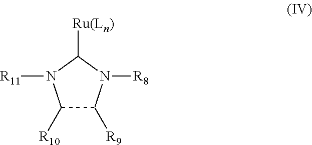

wherein L is a ligand selected from phosphines of Formula (II), halides, C₁-C₆ alkoxides, arene, alkylidine, vinylidine, indenylidine, alkenes, amines, pyridines, phosphine oxides and arsines,
and n is 1 to 5.
and wherein the dotted line denotes a single or double bond.

The term “Lₙ” thus should be understood to mean that according to the present invention, 1, 2, 3, 4 or 5 ligands selected from phosphines of Formula (II), halides, C₁-C₆ alkoxides, arene, alkylidine, vinylidine, indenylidine, alkenes, amines, pyridines, phosphine oxides and arsines may be attached to the Ruthenium atom.

The reaction conditions for the formation of the ruthenium complex of formula (IV) comprises heating for 10-100 minutes at a temperature between 80-200° C., preferably between 90-120° C.

In a specific embodiment of the invention the ruthenium complex of formula (IV) may be isolated. In another embodiment of the invention the ruthenium complex of formula (IV) is generated in situ.

In a preferred embodiment the ruthenium complex of formula (IV) has the following structure 5.1:

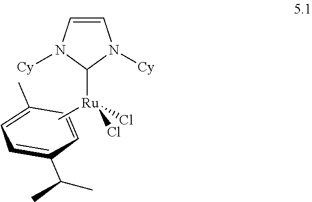

In another preferred embodiment the ruthenium complex of formula (IV) has the following structure 5.2:

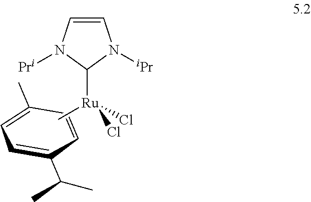

In yet another preferred embodiment the ruthenium complex of formula (IV) has the following structure 5.3:

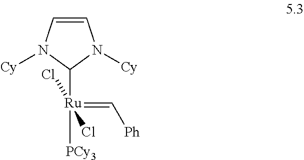

With certain classes of starting materials of formulae A and B and certain ruthenium complexes of formula (IV), said ruthenium complexes may catalyze other chemical reactions which may be carried out either before and/or after the preparation of amides of formula (I) from alcohols of formula A and amines of formula B discussed sofar. Several different well-known classes of reactions such as metathesis, amide formation and hydrogenation may thus be carried out “one-pot” in tandem fashion, all catalyzed by the same specific ruthenium complex of formula (IV). For a discussion of the selectivity of the olefin cross metathesis reaction, see R. H. Grubbs et al., J. Am. Chem. Soc. (2003) 125, 11360.

Thus, in a further embodiment of the invention, the ruthenium complex of formula (IV) is thus capable of first catalyzing a metathesis reaction between an alcohol of formula A′ containing a terminal alkene, and a different alkene of formula C, thereby affording a new alcohol of the general formula A″. Upon adding an amine of formula B to the reaction mixture, the ruthenium complex may subsequently catalyze an amide formation reaction between the alcohol of formula A″ and the amine of formula B, thereby affording an amide of formula (I′). Finally, in the presence of hydrogen, the ruthenium complex may catalyze the hydrogenation of the double bond introduced in the metathesis reaction to afford an amide of formula (I″). These different transformations are illustrated in the following scheme:

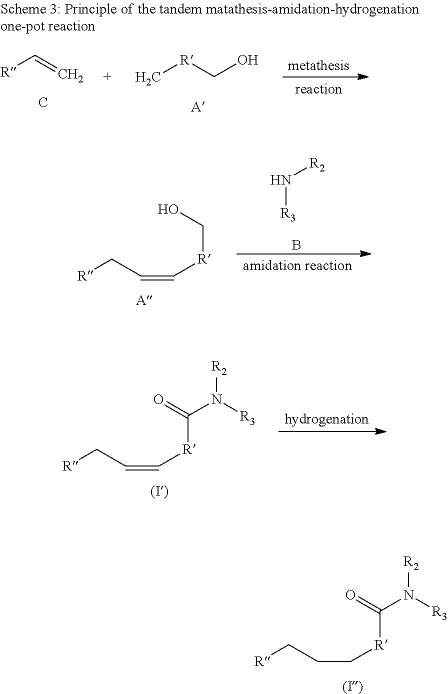

wherein:
R′ is selected from the group consisting of aryl, heteroaryl, C₁-C₂₀ alkyl, alkenyl or aryl-C₁-C₄ alkyl, optionally substituted with one or more substituents selected from halogen, C₁-C₆ alkyl or C₁-C₆ alkoxy, however such that CH₂═CH—R′— is comprised by the definition of R₁
R″ is selected from the group consisting of straight chain or branched C₁-C₂₀ alkyl optionally substituted with one or more substituents selected from halogen, oxygen or aryl
R₁, R₂ and R₃ are as defined previously

It is understood that the invention is not limited to these embodiments set forth herein for illustration, but embraces all such forms thereof as come within the scope of the claims.

### BRIEF DESCRIPTION OF THE DRAWINGS

FIG. 1 demonstrates the effect of the azolium salt on the amidation reaction.

FIG. 2 demonstrates the effect of the phosphine ligand on the amidation reaction.

FIG. 3 demonstrates the substrate scope of the amidation reaction.

FIG. 4 demonstrates the performance of different catalysts

FIG. 5 demonstrates a screening study of metathesis catalysts' performance in the amidation reaction

FIG. 6 demonstrates a screening study of NHC-ligands for amidation with Grubbs 1^{st} generation metathesis catalyst.

FIG. 7 demonstrates the scope of the Ru-complex 5.2 in the amidation reaction.

FIG. 8 demonstrates the scope of the Hoveyda-Grubbs 1st generation metathesis catalyst in the amidation reaction

### DETAILED DESCRIPTION OF THE INVENTION

### Definition of Substituents

As used in the present invention, the term “C₁-C₂₀ alkyl” refers to a straight chained or branched saturated hydrocarbon having from one to twenty carbon atoms inclusive. Examples of such groups include, but are not limited to, methyl, 2-propyl, 1-butyl, 2-butyl, 2-methyl-2-propyl, 2-methyl-1-butyl, 1-hexyl, 1-octyl, 1-decyl and 1-dodecyl.

Similarly, the term “C₁-C₆ alkyl” refers to a straight chained or branched saturated hydrocarbon having from one to six carbon atoms inclusive.

Similarly, the term “C₁-C₄ alkyl” refers to a straight chained or branched saturated hydrocarbon having from one to four carbon atoms inclusive.

As used herein, the term “C₃-C₆ cycloalkyl” typically refers to cyclopropyl, cyclobutyl, cyclopentyl and cyclohexyl.

As used herein, the term “C₁-C₆ alkoxy” refers to a straight chain or branched saturated alkoxy group having from one to six carbon atoms inclusive with the open valency on the oxygen. Examples of such groups include, but are not limited to, methoxy, ethoxy, n-butoxy, 2-methyl-pentoxy and n-hexyloxy.

As used herein, the term “C₁-C₆ alkoxides” refers to the anions of straight chain or branched saturated alkanols having from one to six carbon atoms inclusive with the negative charge on the oxygen atom. Typical examples include methoxide and tert-butoxide.

As used herein, the term “alkene” refers to a straight chained, branched or cyclic hydrocarbon having from one to eight carbon atoms inclusive, and one or two double bonds which may be isolated or conjugated.

As used herein, the term “halide” refers to the anion of a halogen atom.

As used herein, the term “phosphine” refers to compounds of the general formula (II) as defined above. Analogously, as used herein, the term “phosphine oxides” refers to phosphines of the general formula (II) which have been oxidized to their related oxides.

As used herein, the terms “arene” and “aryl” both refer to a mono- or bicyclic aromatic group having from six to twelve carbon atoms inclusive. Examples of such groups include, but are not limited to, phenyl, naphthyl, indenyl, tetrahydronaphthyl and indanyl.

As used herein, the term bi(C₆-C₁₂aryl) refers to two mono- or bicyclic aromatic groups joined by a single bond, each group having from six to twelve carbon atoms inclusive. A typical non-limitating example of such groups is biphenyl.

As used herein, the term aryl-C₁-C₄ alkyl refers to an aryl group as defined above, substituted with a C₁-C₄ alkyl as defined above, with the open valency on the terminal carbon of the C₁-C₄ alkyl group. Examples of such groups include, but are not limited to, benzyl and phenylethyl.

As used herein, the term “arsine” refers to compounds of formula As(R)₃ wherein R may be the same or different and selected from C₁-C₆ alkyl and aryl, as defined above.

As used herein, the term “heteroaryl” refers to a mono- or bicyclic heteroaromatic group having from five to ten carbon atoms and from one to three heteroatoms inclusive, wherein the heteroatoms are selected individually from N, O and S. Examples of such groups include, but are not limited to, pyridyl, 2-thienyl, 3-thienyl, 2-furyl, 3-furyl, quinolinyl and naphthyridyl.

### Abbreviations

As used herein, the following abbreviations mean:

### Bn: Benzyl

### COD: 1,5-Cyclooctadiene

### Cy: Cyclohexyl

### Cyp: Cyclopentyl

ICy: 1,3-Dicyclohexylimidazol-2-ylidene
IⁱPr: 1,3-Di-iso-propylimidazol-2-ylidene
IMe: 1,3-Dimethylimidazol-2-ylidene
IMes: 1,3-Dimesitylimidazol-2-ylidene

### Imid: Imidazole

I^{t}Bu: 1,3-Di-tert-butylimidazol-2-ylidene
KHMDS: Potassium bis(trimethylsilyl)amide

### Me: Methyl

Mes: Mesityl, i.e. 2,4,6-trimethylphenyl
NHC: N-Heterocyclic carbene

### Q: Quantitative

RCM: Ring closing metathesis

Given the widespread importance of amides in biochemical and chemical systems, an efficient synthesis that avoids wasteful use of stoichiometric coupling reagents or corrosive acidic and basic media is highly desirable. The present invention addresses this problem by disclosing a direct, easy, economical and environmentally friendly synthesis of carboxamides of the formula (I), comprising reacting a primary alcohol of formula A with a primary or secondary amine of formula B in the presence of a ruthenium NHC complex of formula (IV):

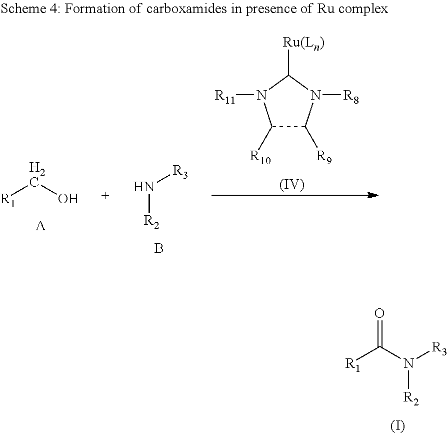

The NHC complex of formula (IV) may be prepared in situ from a phosphine, an azolium salt and a suitable source of ruthenium in the presence of a base and optional solvents.

In one embodiment of the invention, the mixture of the phosphine, the azolium salt, the suitable source of ruthenium and the base is heated to 110° C. for 20 minutes, and an optional solvent is added before the substrates (alcohol and amine) are added. In another embodiment the reaction is conducted in an inert atmosphere.

In another embodiment the reaction between the primary alcohol of formula A with a primary or secondary amine of formula B in the presence of a ruthenium NHC complex of formula (IV) is carried out by stirring the reaction mixture for up to about 24 h at a temperature between 80-200° C. or until deemed complete, using routine analytical methods such as TLC, HPLC or GC, optionally using MS detection. After cooling to room temperature, the reaction mixture is worked up and the product isolated and optionally purified using standard methods (e.g. recrystallization, column chromatography etc.).

Presumably, the reaction proceeds through the intermediate aldehyde which reacts with the amine to give a hemiaminal that is subsequently dehydrogenated to the amide.

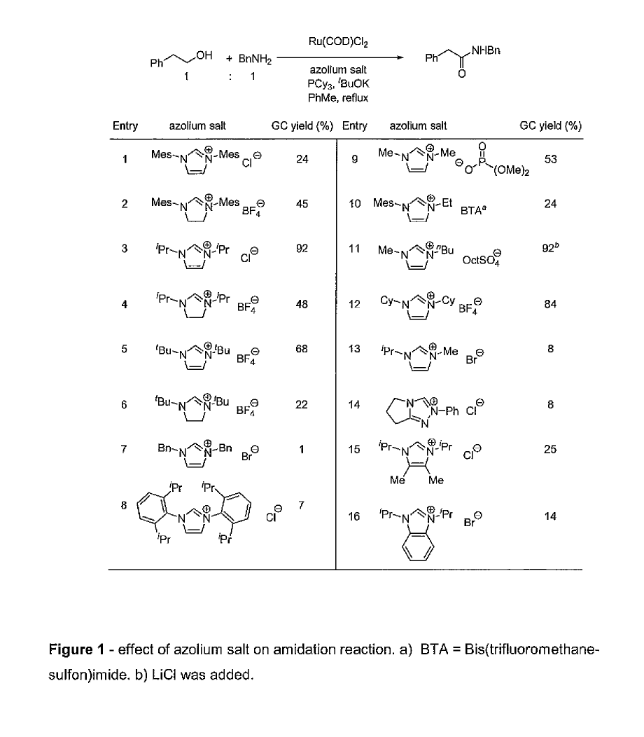

The effect of the azolium salt and hence the ruthenium NHC catalyst was studied, the results of which appear in FIG. 1.

All reactions were carried out using tricyclohexylphosphine (PCy₃) as the phosphine ligand. The best results were obtained with the imidazolium salts according to entries 3, 11 and 12, although several other azolium salts also afforded good yields.

In a specific embodiment the azolium salt is an imidazolium chloride. In another embodiment the azolium salt is an imidazolium tetrafluoroborate. In yet another embodiment the azolium salt is an imidazolium octylsulfate.

In a further embodiment R₈ and R₁₁ are the same. In another embodiment R₈ and R₁₁ are different. In a further embodiment R₈ and R₁₁ are linear or branched C₁-C₂₀ alkyl.

In another embodiment R₉ and R₁₀ are the same. In a further embodiment R₉ and R₁₀ are different. In a further embodiment R₉ and R₁₀ are both hydrogen. In another embodiment one of R₉ and R₁₀ is hydrogen and one of R₉ and R₁₀ is selected from linear or branched C₁-C₆ linear or branched alkyl or aryl.

In a preferred embodiment X⁻ is Cl⁻, R₈ and R₁₁ are both isopropyl, and R₉ and R₁₀ are hydrogen and the dotted line denotes a double bond.

In another preferred embodiment X⁻ is Cl⁻, R₈ and R₁₁ are both methyl, and R₉ and R₁₀ are hydrogen and the dotted line denotes a double bond.

In another preferred embodiment X⁻ is Cl⁻ or BF₄⁻, R₈ and R₁₁ are both cyclohexyl, and R₉ and R₁₀ are hydrogen and the dotted line denotes a double bond.

In another embodiment X⁻ is alkyl-SO₃⁻, R₈ is n-butyl, R₁₁ is methyl, and R₉ and R₁₀ are hydrogen and the dotted line denotes a double bond. In yet another embodiment X⁻ is BF₄⁻, R₈ and R₁₁ are both cyclohexyl, and R₉ and R₁₀ are hydrogen and the dotted line denotes a double bond. In yet another embodiment X⁻ is BF₄⁻, R₈ and R₁₁ are both tert-butyl, and R₉ and R₁₀ are hydrogen and the dotted line denotes a double bond.

In a preferred embodiment of the invention the azolium salt is selected from 1,3-dicyclohexyl-1H-imidazol-3-ium chloride, 1,3-dicyclohexyl-1H-imidazol-3-ium tetrafluoroborate, 1,3-diisopropyl-1H-imidazol-3-ium chloride, 1,3-diisopropyl-1H-imidazol-3-ium tetrafluoroborate, 1,3-dimethyl-1H-imidazol-3-ium chloride and 1,3-dimethyl-1 tetrafluoroborate.

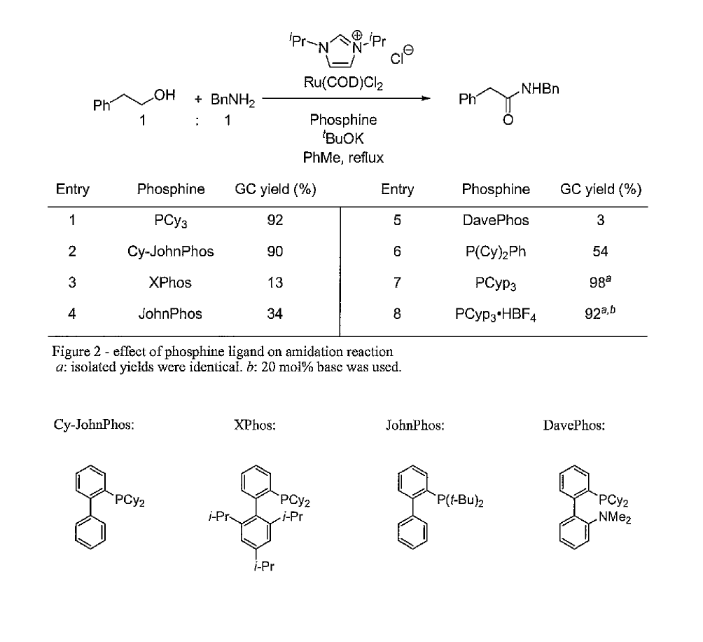

The ruthenium NHC catalyst was further optimized by investigating the effect of the phosphine ligand. The screening was narrowed to include ligands that were similar to the PCy₃. The ligands tested and the results are shown in FIG. 2, Tricyclopentylphosphine (PCyp₃) turned out to be slightly better than PCy₃, but several other phosphines also afforded good yields.

In a preferred embodiment of the invention R₄, R₅ and R₆ are all cyclopentyl. In another specific embodiment R₄, R₅ and R₆ are all cyclohexyl. In yet another embodiment R₄ and R₅ are both cyclohexyl and R₆ is biphenyl.

In another preferred embodiment of the invention the phosphine is selected from tricyclopentylphosphine, tricyclohexylphosphine, biphenyldicyclohexylphosphine (Cy-JohnPhos), or a tetrahydrofluoroborate (HBF₄) salt thereof.

Since trialkylphosphines tend to be easily oxidized, the HBF₄ salts are often used as air stable substitutes. In this case the PCyp₃.HBF₄ gave a slightly lower GC yield than the free phosphine, but the isolated yields turned out to be identical. For practical reasons the HBF₄ salt was employed.

In a specific embodiment of the invention the phosphine of formula (II) is therefore employed as a salt, such as the tetrafluoroborate (HBF₄) salt. In another specific embodiment of the invention the phosphine ligand is tricyclopentylphosphine tetrafluoroborate. In yet another specific embodiment of the invention the phosphine ligand is tricyclohexylphosphine tetrafluoroborate.

The ratios of ruthenium to the ligands and to the base have also been examined, and no improvement was observed when an excess of ligands were used.

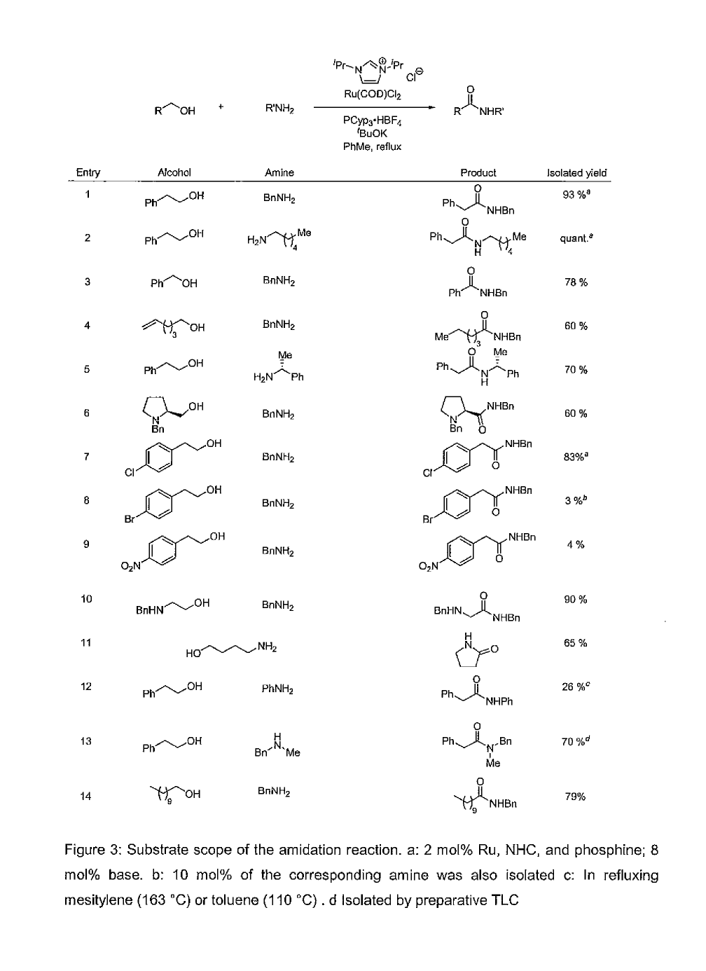

A range of different primary alcohols were reacted with primary amines to afford the corresponding secondary amides in 60-100% isolated yield (FIG. 3, entries 1-7). Sterically unhindered alcohols and amines gave the amide in high yield (FIG. 3, entry 1 and 2). Benzyl alcohol was converted into benzamide (FIG. 3, entry 3) while hex-5-en-1-ol gave hexanamide with concomitant reduction of the olefin (FIG. 3, entry 4). An optically pure amine could be employed and the product showed no sign of racemization according to optical rotation (FIG. 3, entry 5). Optically pure N-benzyl-L-prolinol was converted into N,N′-dibenzyl-L-prolinamide with no sign of epimerization (FIG. 3, entry 6). An aryl chloride performed well (83% yield; 2 mol % catalyst) in the reaction (FIG. 3, entry 7), but the aryl bromide analogue resulted in only 3% yield (along with 10% of the corresponding amine; FIG. 3, entry 8). The nitro group also resulted in a very low yield (FIG. 3, entry 9). Next, it was demonstrated that a primary amine can be coupled in good yield in the presence of a secondary amine (FIG. 3, entry 10). N-Benzylethanolamine could be coupled with benzylamine in high yield (FIG. 3, entry 10) which shows that the transformation is selective for a primary amine. The amidation could also be carried out in an intramolecular fashion as illustrated with the formation of butyrolactam from 4-aminobutan-1-ol (FIG. 3, entry 11). Aniline and secondary amines, on the other hand, did not react with primary alcohols at 110° C. However, when the temperature was raised to 163° C. complete conversion of the alcohol was observed. At this temperature, aniline gave the amide in low yield while the remaining portion of the alcohol underwent self condensation into the corresponding ester (FIG. 3, entry 12), whereas N-methylbenzylamine gave the amide in good yield (FIG. 3, entry 13).

The amidation reaction examples illustrated in FIG. 3 all occur in the presence of an in situ generated catalyst. In a different embodiment of the invention the Ru complexes of formula (IV) can be isolated and used as such in the amidation reaction, optionally in the presence of an added phosphine, rather than being prepared in situ, which will be discussed in the following.

It had been anticipated that the simplest method would be to generate the NHC-ligand in the presence of [Ru(COD)Cl₂]n and isolate the formed catalyst. However, ruthenium-complexes containing both an NHC-ligand and a COD-ligand proved too sensitive to be isolated and applied in the amidation reaction without applying glove-box or Schlenk techniques which would limit their practical use.

Success was achieved by turning to a more stable ligand, i.e. an 18-electron complex with the cymene ligand. The cymene ligand was envisioned to depart from ruthenium at elevated temperature (85° C.) and generate the same catalytically active species, although separate addition of a phosphine ligand would be required [Noels, A. F. et al., Chem. Commun. 2003, 1526-1527]. One-pot silver-carbene formation and NHC-transfer to ruthenium afforded the desired complex in excellent yield after flash chromatography [Louie, J. et al., J. Am. Chem. Soc. (2001) 123 11312-11313] (scheme 5):

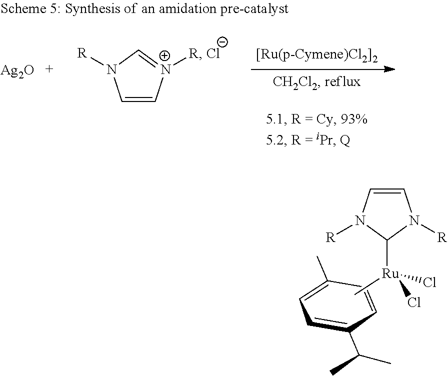

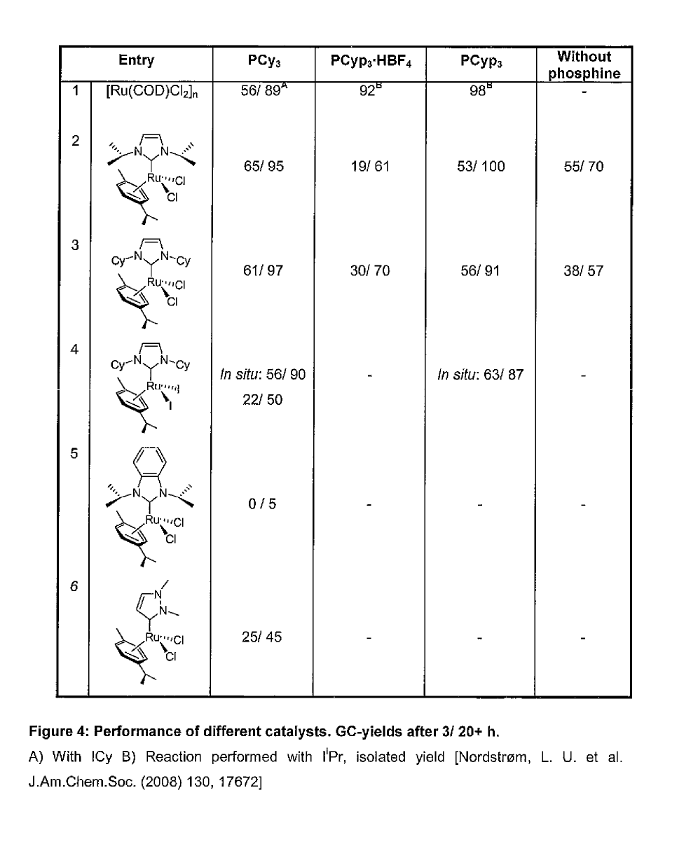

The catalysts of structure 5.1 and 5.2 (see scheme 5) proved to be equally efficient at producing the amide from 2-phenylethanol and benzylamine as the in situ generated catalyst from [Ru(COD)Cl₂]n (scheme 6 and FIG. 4).

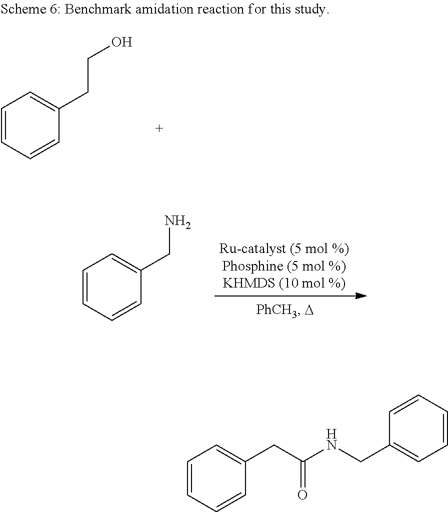

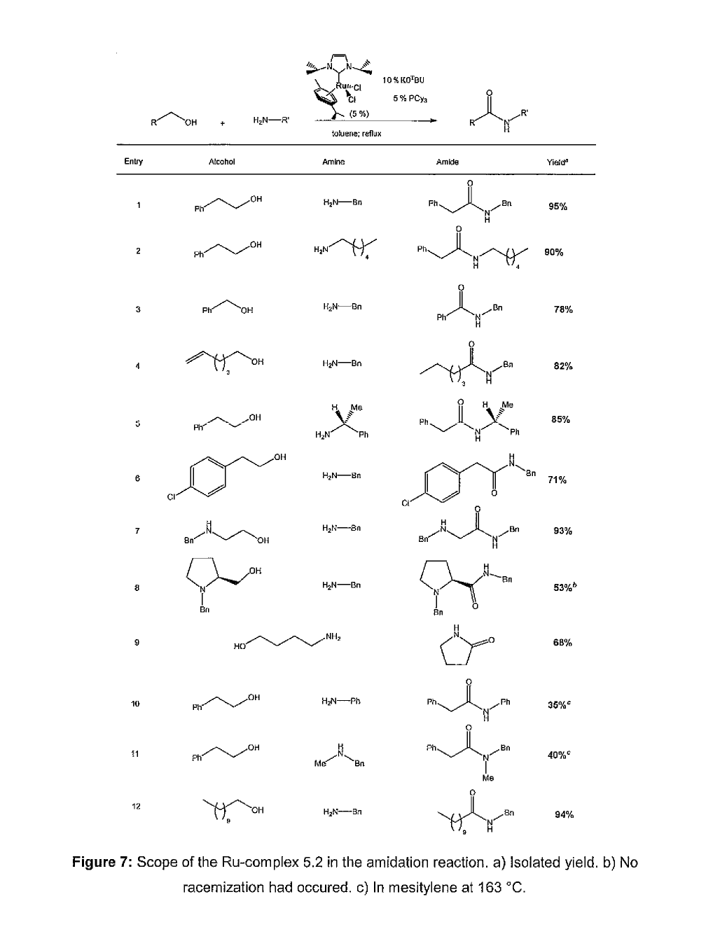

FIG. 4 shows that both the NHC-ligands ICy and IⁱPr (entry 2 & 3) perform equally well as the initial system reported by Madsen et al. [Nordstrom, L. U.; Vogt, H.; Madsen, R., J. Am. Chem. Soc. 2008, 130, 17672-17673] which provided the amide in 56 and 89% yield after 3 and 20+h respectively (FIG. 4, entry 1). The scope of the isolatable Ru-complex 5.2 was further investigated (see FIG. 7).

It was tried to alter different parts of the catalyst in order to improve the system. Exchange of the chlorides bonded to the ruthenium with iodides did not bring about any improvement (compare FIG. 4 entry 3 & 4). Saturated NHC-ligands were found to perform poorly, and an NHC-ligand where the backbone was fused to an aromatic system (FIG. 4 entry 5) was found to generate an almost completely inactive catalyst probably because even if the NHC-ligand is still unsaturated, the electrons are now less available.

Reducing the steric requirements around the ruthenium center was also attempted. An abnormal carbene as shown in FIG. 4, entry 6 was chosen as a suitable testing ground. The complex was synthesized according to the methods recently described by Penis and co-workers [Peris et al., Organometallics 2008, 27, 4254-4259], but the catalyst unfortunately only provided conversion to the desired amide to some extent.

A different, more successful modification was found in the structurally related Ring Closure Metathesis (RCM) catalysts pioneered by Grubbs and co-workers, who reported in 2001 that applying an H₂-atmosphere after ring closing metathesis using Grubbs 1st generation catalyst would generate the effective hydrogenation catalyst RuHCl(H₂)(PCy₃)₂ [Grubbs, R. H. et al., J. Am. Chem. Soc. 2001, 123, 11312-11313]. The authors showed that the benzylidene ligand for starting the RCM had been hydrogenated off. This allowed for the synthesis of a fully elaborated pre-catalyst (with a phosphine and an NHC-ligand) for the amidation reaction that would produce the proper catalytic species, because the hydrogen formed in the reaction would remove the benzylidene group. Indeed, it was found that simply applying Grubbs 1^{st} generation catalyst without any NHC-ligand under standard conditions provided the amide in 71% yield after ˜20 h. However, a vast improvement was observed upon addition of ICyHCl or IⁱPrHCl which allowed the amidation reaction to take place in 76% yield after 3 h and to afford the amide quantitatively overnight.

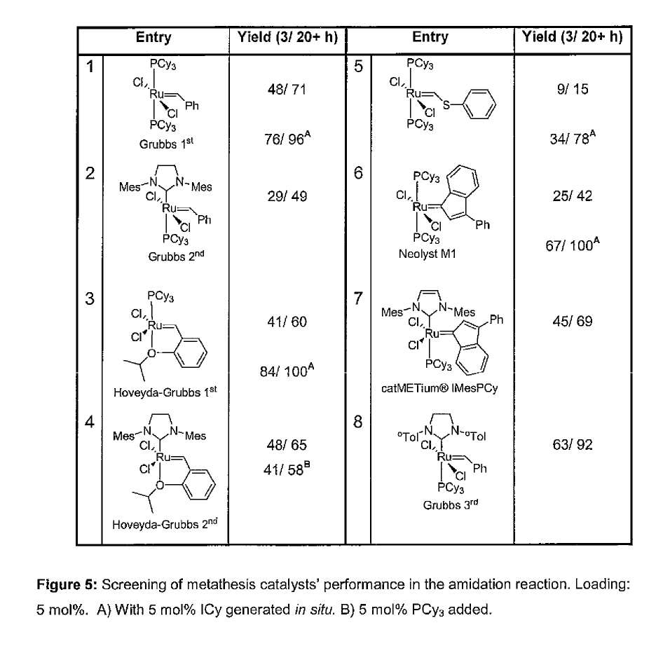

A screening of different metathesis catalysts was subsequently carried out to investigate whether these would afford a ready-to-use catalyst, FIG. 5.

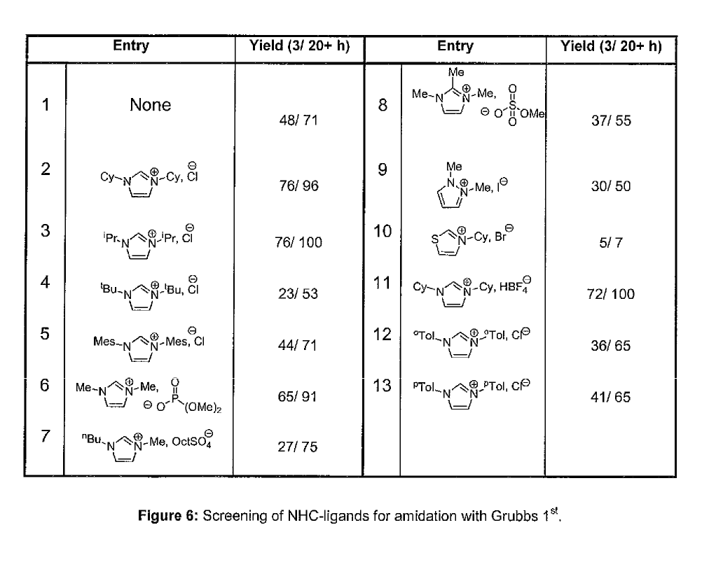

From FIG. 5 it is seen that no metathesis catalyst on its own brings any improvement as regards improved activity in catalyzing the amidation reaction. The newer generation metathesis catalyst (entry 8) did however show comparable reactivity to the complexes containing the cymene ligand, catalysts 5.1 and 5.2 (scheme 5), which was surprising since it contained an NHC-ligand with a saturated backbone. However, by generating ICy in situ with the catalysts without any NHC-ligand (FIG. 5, entry 1, 3 and 6) very effective amidation catalysts were formed. This led to a further small screening of other NHC-ligands being undertaken (FIG. 6) with the conditions from scheme 6.

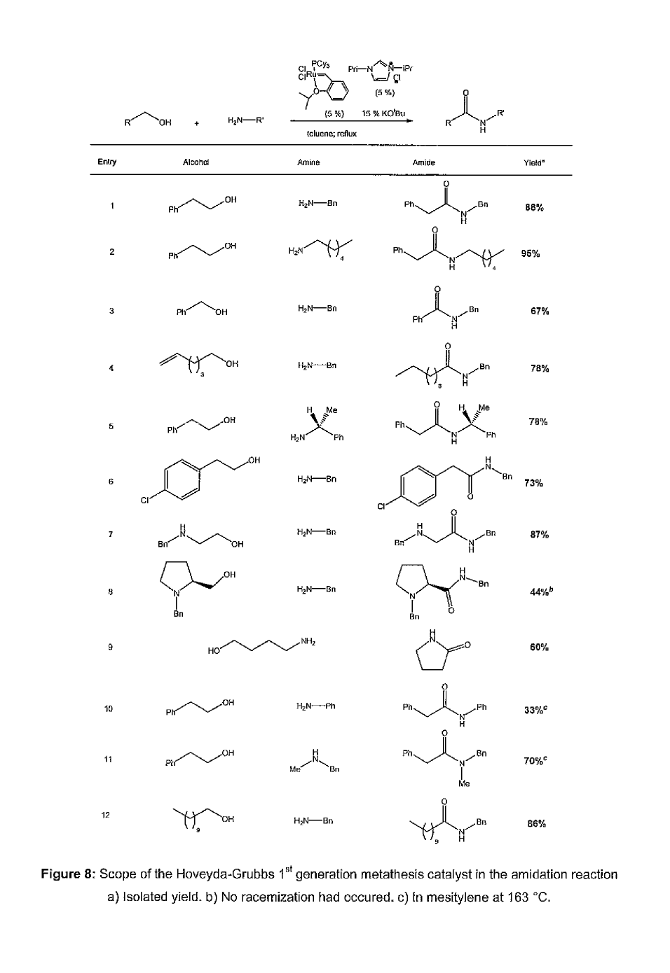

From FIG. 6 it is seen that the cyclohexyl (Cy), i-propyl (ⁱPr) and methyl (Me) substituted NHC ligands proved superior, often affording quantitative yields overnight, but other substituted NHC ligands also afforded reasonable yields of the amidation product. As can be seen from FIG. 6, entry 10, the N-cyclohexyl thiazolium salt quenched the reaction. Most likely the thiazol-2-ylidene dimerizes readily when deprotonated as reported by Arduengo et al. [Arduengo, A. J. I.; Göerlich, J. R.; Marshall, W. J., Liebigs Ann./Recl. 1997, 365-374]. The scope of the Hoveyda-Grubbs 1^{st} generation metathesis catalyst (FIG. 5, entry 3) in the amidation reaction was further investigated (see FIG. 8).

On the basis of the findings in FIG. 5 and scheme 5 the synthesis of the known Grubbs 2^{nd} generation RCM catalyst-analoque, 5.3, was undertaken (scheme 7) [Herrmann, W. A. et al., Angew. Chem. Int. Ed. (1999), 38, 2416-2419; Herrmann, W. A. et al., J. Organomet. Chem. (1999), 582, 362-365]. Following the original procedure reported by Herrmann et al. the isolated solid proved inferior in the amidation reaction. They noted that low temperature was required in order to achieve the mono-carbene complex in THF. Only by applying the conditions reported by Nolan et al. were we able to produce a isolated complex showing similar efficiency as the in situ generated system: 74% yield in 3 h and 97% overnight [Nolan, S. P., Organometallics 2000, 19, 2055-2057; Nolan, S P., Organometallics 2002, 21, 442-444.]

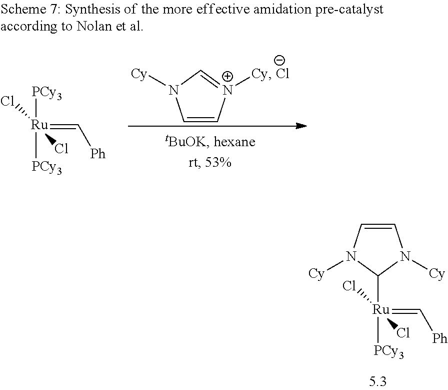

Catalyst 5.3 also produced the desired tertiary amide from 2-phenylethanol and N-benzylmethylamine in toluene in 65% isolated yield.

The realization that metathesis catalysts work well in the amidation reaction has made it possible to achieve a one-pot, tandem reaction whereby alkenes can be coupled with other hydroxy-substituted alkenes in a metathesis reaction affording a new hydroxyalkene. This can subsequently in situ be reacted with an amine to produce an amidoalkene, which ultimately may be hydrogenated to remove the installed double bond.

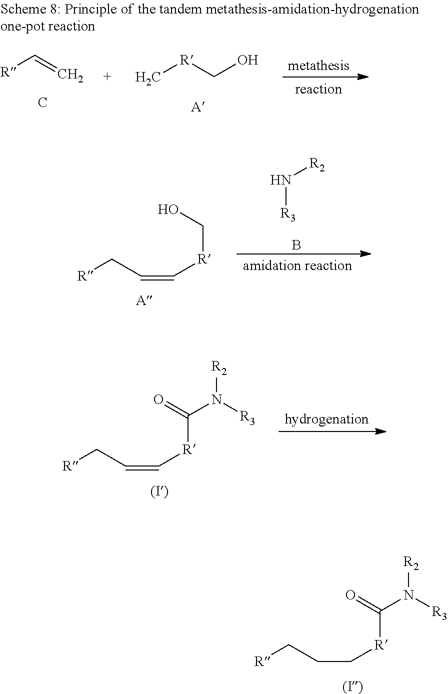

The invention disclosed herein is further illustrated by the following non-limiting preparative examples:

### EXPERIMENTAL

### General Comments:

All chemicals were obtained form Sigma-Aldrich and used without further purification, except toluene which was dried over sodium and distilled under a nitrogen atmosphere. PCyp₃.HBF₄ was prepared according to a known procedure.¹ Mass spectrometry was performed by direct inlet on a Shimadzu GCMS-QP5000 instrument. IR spectra were recorded on a Bruker alpha-P spectrometer. ¹H and ¹³C NMR spectra were obtained on a Varian Mercury 300 instrument at 300 MHz and 75 MHz, respectively. The spectra were calibrated using residual solvent signals. GC yields were measured on a Shimadzu GC2010 equipped with an Equity™ 1 column and with dodecane as an internal standard. Column chromatography was performed on silica gel (220-440 mesh). ¹ Zhou, J.; Fu, G. C. J. Am. Chem. Soc. 2003, 125, 12527-12530.

### Preparation of N-benzyl 2-phenylacetamide

Ru(COD)Cl₂ (7.0 mg, 0.025 mmol), PCyp₃.HBF₄ (8.2 mg, 0.025 mmol), 1,3-diisopropylimidazolium chloride (4.7 mg, 0.025 mmol), and ^{t}BuOK (11.2 mg, 0.10 mmol) were weighted into an oven-dried Schlenk tube. A condenser was attached and vacuum applied before the flask was filled with argon (repeat twice). Freshly distilled toluene (1 mL) was then added and the mixture was heated to reflux temperature for 20 min. The flask was removed from the oil bath and 2-phenylethanol (1.25 mmol, 153 mg, 150 □L) and benzylamine (1.25 mmol, 134 mg, 137 □L) were added. The flask was returned to the oil bath for 24 hours. After cooling to room temperature, the solvent was removed in vacuo and the residue was purified by column chromatography (Eluent: pentane/ethyl acetate 4:1→1:1) to give N-benzyl 2-phenylacetamide (261 mg, 1.16 mmol, 93%) as a white solid.

IR (KBr): 3288, 3063, 3030, 1637, 1551, 1454, 1431, 1029, 693, 602 cm⁻¹.

Mp. 118-119° C. Lit²: 118-119° C. ² Choi, D.; Stables, J. P.; Kohn, H. Bioorg. Med. Chem. 1996, 4, 2105-2114.

¹H NMR (CDCl₃): □ 7.38-7.15 (m, 10H, Ar), 5.88 (bs, 1H, —CONH—), 4.40 (d, 2H, J=5.8 Hz, N—CH₂-Ph), 3.61 (s, 2H, Ph-CH₂—CO) ppm.

¹³C NMR (CDCl₃): □ 171.0 (C═O), 138.2, 134.9, 129.5, 129.1, 128.7, 127.6, 127.5, 127.5 (Ar), 43.9, 43.6 (2×—CH₂—) ppm.

MS: m/z 226 [M+H].

### General Amidation Procedure Using an Isolated Ru-Complex

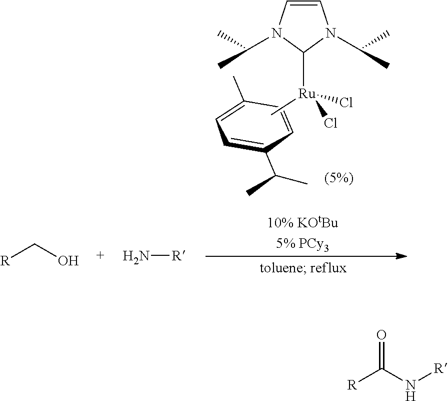

The isolated Ru(p-cymene)Cl₂IⁱPr complex (0.025 mmol), PCy₃ (0.025 mmol) and KOtBu (0.05 mmol) were weighted into an oven-dried Schlenk tube. A condenser was attached and vacuum applied before the flask was filled with argon (repeat twice). Freshly distilled toluene (1 mL) was then added and the mixture was heated at reflux temperature for 20 min. The flask was removed from the oil bath and the alcohol (0.5 mmol) and the amine (0.5 mmol) were added. The flask was returned to the oil bath and heated at reflux for 24 hours. After cooling to room temperature, the solvent was removed in vacuo and the residue was purified by column chromatography to give the amide. Results are presented in FIG. 7.

### General Amidation Procedure Using Metathesis Catalysts:

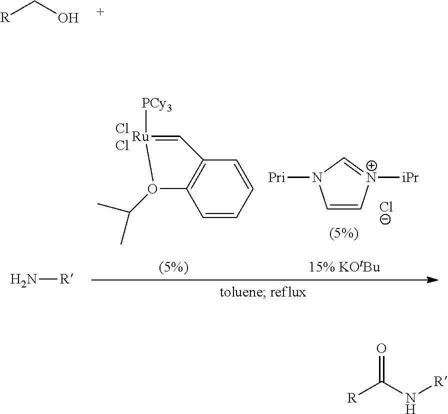

Hoveyda-Grubbs 1^{st} generation catalyst (0.025 mmol), 1,3-diisopropylimidazolium chloride (0.025 mmol) and KOtBu (0.075 mmol) were weighted into an oven-dried Schlenk tube. A condenser was attached and vacuum applied before the flask was filled with argon (procedure repeated twice). Freshly distilled toluene (1 mL) was then added and the mixture was heated at reflux temperature for 20 min. The flask was removed from the oil bath and the alcohol (0.5 mmol) and the amine (0.5 mmol) were added. The flask was returned to the oil bath and heated at reflux for 24 hours. After cooling to room temperature, the solvent was removed in vacuo and the residue was purified by column chromatography to give the amide. Results are presented in FIG. 8.

1.-15. (canceled)

16. A method for preparing amides of formula (I) from alcohols of formula A and amines of formula B in the presence of a ruthenium complex of formula (IV): 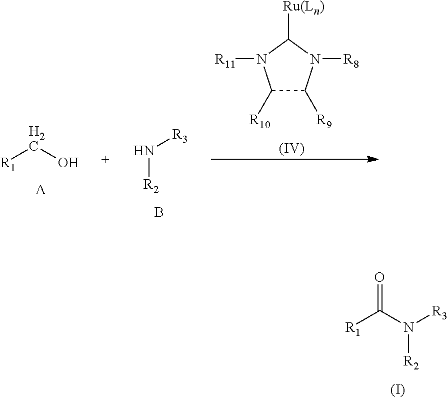
wherein
R1 and R2 are individually selected from the group consisting of hydrogen, aryl, heteroaryl, C1-C20 alkyl or aryl-C1-C4 alkyl, optionally substituted with one or more substituents selected from halogen, C1-C6 alkyl or C1-C6 alkoxy, and wherein, in the amide of formula (I), R1 and R2 may be connected with a single bond,
R3 is selected from hydrogen and C1-C4 alkyl,
R8 and R11 can be the same or different and are independently selected from the group consisting of C1-C6 linear or branched alkyl, C3-C6 cycloalkyl, aryl, heteroaryl,
R9 and R10 are independently selected from the group consisting of hydrogen, C1-C6 linear or branched alkyl and aryl, and wherein R9 and R10 together with the heterocyclic ring they are attached to may form a 5-7 membered saturated ring optionally containing one or two heteroatoms selected from oxygen or nitrogen,
L is a ligand selected from phosphines, halides, C1-C6 alkoxides, arenes, alkylidine, vinylidine, indenylidine, alkenes, amines, pyridines, phosphine oxides and arsines,
n is an integer from 1 to 4
the dotted line in formula (IV) denotes a single or double bond,
which method comprises
mixing the substrates of formula A and formula B in the presence of the catalyst of formula (IV) to form a reaction mixture,
optionally adding a phosphine and/or a solvent, and
heating the reaction mixture until the reaction is deemed complete.

17. The method according to claim 16, further comprising a purification step.

18. The method according to claim 16, wherein the ruthenium complex of formula (IV) is prepared in situ by a method comprising heating a mixture comprising
a phosphine
an azolium salt
a base
a ruthenium (II) source and
an optional solvent
for 10-100 minutes at a temperature between 80-200° C.

19. The method according to claim 18, wherein the mixture is heated at 90-120° C.

20. The method according to claim 18, wherein ruthenium complex mixture is heated at approximately 110° C. for 15-40 minutes before the substrates of formula A and B are added to form the reaction mixture.

21. The method according to claim 16, wherein L is a phosphine of formula (II) 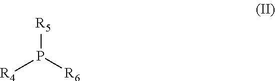
wherein R4, R5 and R6 are independently selected from the group consisting of C1-C6 linear or branched alkyl, C3-C6 cycloalkyl, C6-C12 aryl, C6-C12 aryl-C1-C4 alkyl, heteroaryl, bi(C6-C12 aryl), wherein each C6-C12 aryl and heteroaryl may optionally be substituted with up to three substituents selected from the group consisting of halogen, C1-C4 alkyl and C1-C4 alkoxy, and wherein two substituents selected from R4, R5 and R6 may be fused and together with the phosphorous atom form a 5-7 membered ring.

22. The method according to claim 18, wherein the azolium salt is of the formula (III) 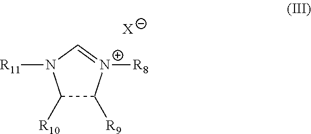
wherein X— is selected from the group consisting of Cl—, Br—; I—, BF₄—, CF₃SO₃—, C1-C6-alkyl-SO₃— and (C1-C6 alkoxy)₂P(═O)O—
and wherein R8 and R11 are independently selected from the group consisting of C1-C6 linear or branched alkyl, C3-C6 cycloalkyl, C6-C12 aryl, and heteroaryl, and
wherein R9 and R10 are independently selected from the group consisting of hydrogen, C1-C6 linear or branched alkyl and C6-C12 aryl, and wherein R9 and R10 together with the heterocyclic ring they are attached to may form a 5-7 membered saturated ring optionally containing one or two heteroatoms selected from oxygen or nitrogen,
wherein each C6-C12 aryl may be substituted with up to three substituents selected from the group consisting of halogen, C1-C4 alkyl and C1-C4 alkoxy,
and wherein the dotted line denotes a single or double bond.

23. The method according to claim 18, wherein the base is selected from the group consisting of alkali metal C1-C6 alkoxides, alkali metal carbonates and alkali metal amides.

24. The method according to claim 18 wherein the base is selected from the group consisting of potassium t-butoxide, cesium carbonate and potassium bis(trimethylsilyl)amide.

25. The method according to claim 18, wherein the ruthenium source is selected from the group consisting of Ru(PPh₃)3Cl₂, Ru(alkene)Cl₂ and Ru(arene)Cl₂, wherein the alkene ligand is selected from norbornene, ethene, cyclooctene (COE) and cyclooctadiene (COD), and wherein the arene ligand is selected from benzene, toluene, mesitylene, p-cymene and naphthalene.

26. The method according to claim 18 wherein the ruthenium source is Ru(COD)Cl₂.

27. The method according to claim 18 wherein the ruthenium source is [Ru(p-Cymene)Cl₂]₂

28. The method according to claim 18 wherein the azolium salt is selected from the group consisting of 1,3-dicyclohexyl-1H-imidazol-3-ium chloride, 1,3-dicyclohexyl-1H-imidazol-3-ium tetrafluoroborate, 1,3-diisopropyl-1H-imidazol-3-ium chloride, 1,3-diisopropyl-1H-imidazol-3-ium tetrafluoroborate, 1,3-dimethyl-1H-imidazol-3-ium chloride, 1,3-dimethyl-1H-imidazol-3-ium tetrafluoroborate.

29. The method according to claim 16 wherein L is a phosphine selected from the group consisting of tricycolopentylphosphine, tricyclohexyl-phosphine, biphenyldicyclohexylphosphine (Cy-JohnPhos), or a tetrahydrofluoroborate (HBF4) salt thereof.

30. The method according to claim 16 wherein the ruthenium complex of formula (IV) is selected from one of the following compounds of structure 5.1, 5.2 or 5.3:

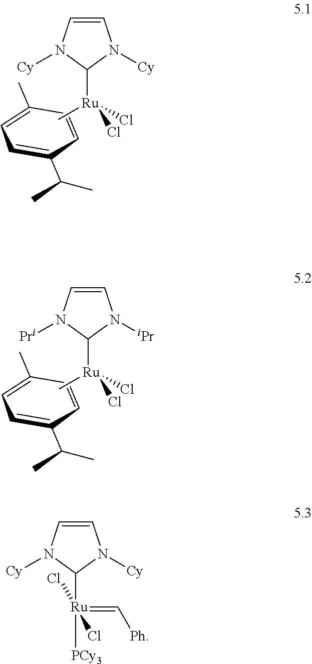

31. The method according to claim 16, wherein the optional solvent is selected from the group consisting of benzene, toluene, xylene, mesitylene, chlorobenzene, dichloromethane, carbon tetrachloride, 1,2-dichloroethane, diethylether, di-n-propylether, di-n-butylether, methyl-tert-butylether (MTBE), tetrahydrofuran (THF), methyltetrahydrofuran, 1,4-dioxane, 1,2-dimethoxyethane (DME), and mixtures thereof.

32. The method of claim 16, further comprising following the progress of the reaction between the substrates of formula A and formula B with an analytical method selected from the group consisting of thin layer chromatography (TLC), gas-liquid chromatography (GLC) or high performance liquid chromatography (HPLC), optionally coupled with mass spectrometrical (MS) detection.
